(19) United States
(12) Patent Application Publication
(10) Pub. No.: US 2002/0107643 A1
(43) Pub. Date: Aug. 8, 2002

(54) PROCESS FOR PAN-GENOMIC DETERMINATION OF MACROMOLECULAR ATOMIC STRUCTURES

(76) Inventors: WAYNE A. HENDRICKSON, NEW YORK, NY (US); BARRY HONIG, HASTINGS, NY (US)

Correspondence Address: JOHN P WHITE, COOPER & DUNHAM LLP, 1185 AVENUE OF AMERICAS, NEW YORK, NY, 10036

(21) Appl. No.: 09/235,986

(22) Filed: Jan. 22, 1999

(51) Int. Cl. G06F 19/00; G01N 33/48; G01N 15/06

(52) U.S. Cl. 702/27; 702/19; 422/68.1

(57) ABSTRACT

A process for pan-genomic determination of three-dimensional macromolecular atomic structures uses a unique combination of components. All known structural information, sequence information and functional information are systematically organized into a genomics database. Advanced tools of bioinformatics are used to cluster all known gene products into families of homologous sequences. Simultaneously, in parallel for each such family, a few cDNAs from appropriately representatives species are cloned into expression vectors for a few expressions systems. Constructs are then screened for expression, and those that are effective advance to the preparative step. Expressed proteins are prepared, purified and characterized. Purified proteins are set to crystallize in parallel against crystallization screens. Crystals that grow are tested for suitable diffraction characteristics. A suitable crystal is frozen, and diffraction data are measured using the multiwavelength anomalous diffraction (MAD) method at a synchrotron which uses undulator beamlines for high-throughput crystallography. Diffraction data are analyzed by the MAD phasing method, an atomic model is built, and the model is refined against the diffraction data. The refined model is analyzed in the context of (1) sequence information from other family members, (2) all other known 3D structures, and (3) functional motifs. It is also analyzed for surface characteristics with the aim to define active sites and macromolecular contact sites. For relevant structures, the active site properties are used to define classes of compounds predicted to have binding potency. Computational tools for homology model building are used to develop models for homologs. The homology models may be used in target selection, drug design, or design of more appropriate constructs for experimental analysis. The ensemble of all known structures is used to further advance the effectiveness of the bioinformatics tools.

### BACKGROUND OF THE INVENTION

Recent developments in genetic analyses and genome sequencing projects provide compelling evidence of a fundamental unity of all life. For example, it has been demonstrated that most human genes have homologs in, for example, mice, worms, and sometimes even microorganisms. In addition, many proteins in an individual organism are related to one another. While there may be 100,000 human genes and over 19,000 protein-coding genes in C. elegans, it is believed that there are on the order of 10,000 distinctive protein modules in all of life on earth. The actual number depends on the granularity level of similarity.

Complete genome sequences are now known for many microorganisms, and for one multicellular organism, the nematode C. elegans. Further, the human genome sequencing project is well underway. Some commercial ventures have determined sequences from the coding regions of nearly all human genes. Several academic and commercial ventures in functional genomics are in progress to map patterns of gene expression so as to gain insight into the functions of gene products.

Up to a few years ago, scientists debated the merits of whole genome sequencing. Since the first whole genome of a free-living organism was sequenced four years ago, however, genomics, i.e., the science based on the sequencing of whole genomes, has revolutionized the approach to many of the most important questions in basic biology and medicine. Sequence-based genomics has enabled exhaustive arrangement of proteins, across genomes and across species, into classes. The ongoing successes in the studies of genome-wide DNA sequences provide valuable insights into biology and considerable commercial potential. However, even greater insight and greater commercial potential can be derived from the gene products, notably protein molecules, which are the entities that actually effect biological action. Structure determination at the atomic level lags far behind, but the accumulated results make it clear that patterns of folding are recurrent and that many proteins have a modular construction. Proteins fall into families of structure as well as function. Estimates vary widely, but the number of unique folds is probably only a few thousand. Only a few hundred of these are now known. A systematic and expeditious method for analyzing the unknown structures would have commercial as well as scientific value.

While genomic sequence information is certainly valuable, it is only one-dimensional and therefore somewhat limited. Genomics based on linear sequence data has limitations on its value in understanding the three-dimensional universe inhabited by biological molecules. It is only as linear sequences are folded into their corresponding three dimensional (3D) structures that they are biologically active and become targets for pharmaceuticals, herbicides or other biotechnological products. Currently, there is little integration between structural and genomic information. Therefore, genomic-driven target identification generally is not influenced by structure.

Understanding biochemical and cellular processes is greatly advanced by knowledge of the three-dimensional atomic structures of proteins and other biological macromolecule. Three dimensional structural information is an important component in, for example, the design of drugs in which genomic information is used in target identification and combinatorial chemistry influences lead discovery. Drug researchers experimentally determine the structure of a target, if possible with a bound inhibitor, and use the structural information to guide the synthesis of new compounds. Alternatively, drug researchers may use the structural properties of known inhibitors or of the binding site itself to search chemical databases for new drug candidates which possess the requisite size, shape and chemical and physical properties that lead to binding.

The use of genomics in target identification and combinatorial chemistry in lead discovery, until now, have not regularly been influenced by structure. However, since it is known in specific cases that structural knowledge can be used in target identification and validation, drug assays and screens, selection of lead compounds, and in designing combinatorial libraries, structure oriented approaches would likely play an increasing role when a comprehensive database of structural information that integrates such uses of structure with genomics is made available. Structure determination using conventional techniques, while being very useful, has the drawback that it is much more costly than sequence determination.

These limitations of sequence-based genomics and conventional structural determination techniques may be removed by the new science of structural genomics. Structural genomics provides the science of structural biology with the same kind of panoramic understanding that sequence genomics has added to the linear information content of the genome. It has been suggested that structural genomics requires a comprehensive structural database that includes the approximately 100,000 expressed proteins thought to be encoded by the human genome (so-called ‘proteome’). While solving all of these structures seems a herculean task, accomplishment of the task may allow us to learn more about the proteins of, for example, bacteria, yeast, archaea and plants. As has been amply illustrated by sequence genomics, there are numerous uses for a comprehensive database of structures.

The information to be gained from structural genomics has a fundamentally different character than information provided by traditional structural biology, and would provide substantial insights into unexpected biological relationships and understanding of the protein motifs or folds of interest in specific biological problems, which would enhance our ability to undertake traditional in-depth structural studies.

Structural biologists traditionally have addressed problems that present important questions of biological function that may best be answered through a structural understanding of the molecular actors. This requires not only structure determination, but also deep analysis with respect to the particular functional question. Structural genomics may be an important tool to such an endeavor. While the accuracy of computational structural predictions would improve with the advent of a comprehensive class database, it has been suggested that the point at which these approaches will be implemented and actually replace experimental structure determination is remote.

In addition to advances in genome sequencing, there have also been advances in the technology for structure determination, such as crystallography, and for sequence and structure analysis, such as bioinformatics. These advances when coupled with rapidly evolving gene sequence information provide a tool for comprehensive studies of the structural underpinning for biology, including commercial applications such as drug discovery.

Bioinformatics refers to the discipline that employs computing systems and computational solution techniques to analyze biological information and data obtained by experiments, modeling, database search, and instrumentation. Bioinformatics includes the use of new computational methods for systematic analysis of genomic and structural data. In addition to widely used sequence analysis programs such as BLAST, a new generation of “advanced” tools have recently become available. Use of these tools has led to significant improvements in the identification of remote sequence homologs. Sequence analysis methods suffer, however, from the fundamental limitation that many proteins with similar functions have no obvious sequence identity.

Three dimensional structure information provides the ultimate solution to this problem. Proteins of similar amino-acid sequence invariably have similar 3D structures and related biological functions. Moreover, it often happens that protein structures are alike even when their sequences are unrelated by conventional methods of comparison. “Fold recognition” methods use structural information to identify relationships between proteins with very different sequences. These methods have been only partially successful, in part because the database of structural paradigms is sparsely populated.

Determination of the structure of a representative member for each and every family may provide a comprehensive view, at some level, of all expressed proteins. The protein families may comprise whole proteins, domains or sequence motifs that may or may not correspond to independent modules. With all protein families accessible, integral membrane proteins, for example, may eventually succumb to mass structure determination. A family-based structural database would provide data for determining the behavior of the proteins, and thereby provide an invaluable resource for improving understanding of protein folds adopted in nature, with the exception of families that would not yield to structure determination, of course. The database would also provide information for bringing to light new functional insights through structural analysis.

Analogous to identification, in sequence genomics, of a protein kinase by recognition of a signature sequence motif, structural genomics may achieve the same objective by examining homology in three dimensions, which would be more powerful than sequence-based approaches. Therefore, one likely product of structural genomics would be identification of ‘surprise’ structural, and in some cases functional, homologies, which could not be identified on the basis of sequence alone. This function of structural genomics may elucidate unexpected links in biological pathways that might have been impossible, or at least very difficult, to determine by using traditional hypothesis-driven methods.

The unsolved members, which probably constitute the majority, of each family may be visualized by homology modeling, based on the known structures of family representatives. Through homology modeling, the 3D structure from one family member can then be used to predict useful models for other family members. These models, constructed with the benefit of the relatively large structural database, would be better than have been achieved using conventional techniques, and provide the foundation for modeling techniques such as secondary structure prediction.

X-ray crystallography is a technique for producing atomic-level 3D structures of biological macromolecules such as proteins. The intensities of X-rays diffracted by crystals can be measured accurately, and the 3D patterns of diffracted intensities are transformed into 3D molecular images. For patterns corresponding to 3 Å resolution and finer, the atomic positions are defined with an accuracy of a few tenths of Angstrom units, to within fractions of bond lengths. Even X-ray diffraction patterns of crystals of large macromolecular assemblages such as viruses or ribosomes may be amenable to analysis. Other techniques, such as nuclear magnetic resonance spectroscopy and electron microscopy, alternatively may be used for structure determination. However, these other techniques have not shown the large scale potential that is available with X-ray crystallography.

X-ray methods are generally more time-consuming than sequencing methods. 3D structure determination still lags far behind genomic sequencing. However, recent advances in the instrumentation and methods of X-ray crystallography provide an opportunity for dramatic enhancement in the rate of structure determination. Notable developments, each maturing within the past few years and requiring or having their most dramatic impact at synchrotron radiation sources, include (1) undulator insertion devices, (2) charge-coupled device (CCD) detectors, (3) cryoprotection of crystals, (4) multiwavelength anomalous diffraction (MAD) phasing methods, and (5) selenomethionyl proteins. These recent technical advances equip crystallography for the task of large-scale structure determination.

Undulators are magnetic arrays in third-generation synchrotrons that produce incredibly bright, laser-like beams of X-rays. The new generation of synchrotron radiation sources enable rapid crystallographic structure determination. Focused undulator beams from the Advance Photon Source (APS) at Argonne National Laboratory have a flux 100-fold greater than its own bending-magnet beams or those of secondgeneration sources such as the National Synchrotron Light Source (NSLS) at Brookhaven. The electronic detectors which are used must be able to cope with such fluxes. Appropriate CCD detectors of adequate size have become available in the last year. For example, 2K by 2K CCD arrays are available from many vendors.

Cryoprotection by flash freezing preserves crystals against radiation damage. The procedures for transfer into cryosolvents have only been perfected in the last few years. Cryoprotection is essential for work with micro-crystals (10-50 micron cross section) which undulators offer. Crystal freezing has had an impact on broadening the range of applicability of X-ray experiments, and particularly for MAD which requires copious amounts of data. Even fairly poor crystals are now within the reach of experiments that once may have produced useful data only for the best capillary mounted crystals.

Phase evaluation by the MAD method, which greatly simplifies structure determination, just came into its own in 1994. MAD requires synchrotron radiation and is enhanced with the excellent energy resolution of an undulator. Coupled with MAD, the routine ability to incorporate selenomethionine systematically into recombinant proteins is transforming the way crystal structures are solved. MAD phasing of selenomethionyl proteins may become the main structure determination method of structural genomics. Selenomethionyl proteins can be expressed easily in most recombinant expression systems, obviating the often tedious stage of search for isomorphous derivatives.

Undulator beamlines provide very brilliant X-rays at energy resolutions appropriate for MAD experiments. Coupled with the use of the newest generation of CCD detectors, a single MAD experiment, which provides all the data necessary for a structure solution, would be obtainable in hours or even a fraction of an hour, rather than several days, which had been the norm.

Other recent advances have very recently brought structural genomics into the realm of the possible. The first of these, as mentioned above, is sequence-based genomics. This has enabled the intelligent classification of protein sequences within and across genomes, thus providing a means to generate a putative list of targets.

It has been proposed that in order to express these proteins one should do the easy things first. For example, if bacterial family members exist, focus on these for expression in bacteria, and if proteins from, for example, thermophiles can be expressed in Escherichia coli, substantial purification can usually be achieved by boiling the recombinant cell extract. Protein classes without identifiable bacterial homologies may be tried in bacterial expression systems, but may ultimately require eukaryotic systems. This ‘easy ones first’ approach may lead to an early focus on relatively small proteins that, based on their sequences, are likely to be soluble. Multi-domain protein and single-pass transmembrane proteins are likely to pose new questions of domain definition that can be addressed first by analytical sequence-based methods, and second by expression trials, limited proteolysis and mass spectrometry studies. Integral membrane proteins would probably await advances enabling better approaches to crystallization or perhaps structure determination by NMR spectroscopic methods.

The family-based approach provides the enormous advantage, over the classical one, that if a protein proves to be a difficult target, we can drop it in favor of another member of its family that proves to be easier. It has also been proposed to undertake parallel studies on multiple family members, at least through the expression and crystallization stages, following through only on those that work easily. Parallel studies coupled with the continual technical advancement of structure determination methods provide ample reason for optimism of significant reductions in the time of the studies.

Structural genomics, for the most part, is still at the planning stage. Some have suggested that it is still unclear what can be learned from structural genomics and whether three-dimensional structures would provide only an incremental advance over sequence-based knowledge. Other unknowns include how a comprehensive structure database can be integrated with other tools to provide new insight.

### SUMMARY OF THE INVENTION

It is an object of the present invention to provide a system and process for the comprehensive analysis of structures representative of those from all forms of life.

It is another object of the present invention to provide a system and process for producing atomic-level structural paradigms representing all major protein families in all forms of life, at a high throughput.

It is another object of the present invention to provide a system and process for producing a comprehensive database of structural information that integrates uses of structure with genomics for expanding the applicability of combinatorial approaches.

It is a further object of the present invention to provide a system and process for producing a comprehensive database of structural information, integrating the uses of structure with genomics, that broadly covers as many gene families as possible, while providing detailed structural information within each family. The database also provides functional insights with detailed surface descriptions, conservation patterns and active sites. The information may be accessed by specifying a molecular name, a gene family name, a protein family or protein name, a metabolic pathway or a particular sequence. All of the information associated with the molecule of interest, including 3D structures, all related proteins, and links to other databases may be obtained from the database. This wealth of information may be used in many ways, including target identification and validation, lead discovery, and design of drug assays, screens, and combinatorial libraries.

A process for pan-genomic determination of three-dimensional macromolecular atomic structures, according to the present invention may include the following steps:

(1) organizing systematically all known structural information, including proprietary structures determined by the process and all other known structures, into a user friendly database, and updating the database with additional structural, sequence, and/or functional information as the information is acquired;

(2) using advanced tools of bioinformatics to cluster all known gene products into families of homologous sequences;

(3) cloning simultaneously, in parallel for each such family, a few cDNAs from appropriately representative species into expression vectors for a few expressions systems;

(4) screening constructs for expression, and those that are effective advance to the preparative step;

(5) preparing, purifying and characterizing expressed proteins;

(6) crystallizing purified proteins in parallel against crystallization screens;

(7) testing crystals that grow for suitable diffraction characteristics;

(8) freezing a suitable crystal, and measuring diffraction data using a multi-wavelength anomalous diffraction method at a synchrotron storage ring which uses undulator or other beamlines designed specifically for high-throughput crystallography;

(9) analyzing the diffraction data by a multi-wavelength anomalous diffraction phasing method or by another technique, building an atomic model, and refining the model against the diffraction data;

(10) analyzing the refined model in a context of sequence information from other family members and in a context of all other known 3D structures, and analyzing for functional motifs (i.e. geometrical disposition of functionally important residues in space) and for surface characteristics with an aim to define active sites and macromolecular contact sites;

(11) for relevant structures, defining classes of compounds predicted to have binding potency using the active site properties information, for example, the GRASP program;

(12) developing models for homologs using computational tools for homology model building; and

(13) using the homology models for target selection, drug design, and/or design of more appropriate constructs for experimental analysis,

(14) using the ensemble of all known structures to further advance the effectiveness of the bioinformatics tools.

### BRIEF DESCRIPTION OF FIGURES

FIG. 1 is a block diagram of one embodiment of a system of the present invention.

FIG. 2 is a diagram showing a process of the present invention.

FIG. 3 is a diagram showing exemplary uses of the structural genomics database.

### DETAILED DESCRIPTION OF THE INVENTION

The present invention provides a tool for direct exploitation of structural information to deduce protein function. A comprehensive database including detailed descriptions of surface properties of both experimentally determined and homology modeled structures is developed. The information in turn is used to identify new sequence/structure/function relationships. The three dimensional structure of a protein is studied to obtain insights as to what its normal function may be, how it may perform its biochemical action, and with what biological pathway it may be associated. In addition, the accumulated body of structural evidence is studied for suggestions of characteristic patterns on protein surfaces (electrostatics, curvature, etc.) that provide insights into function.

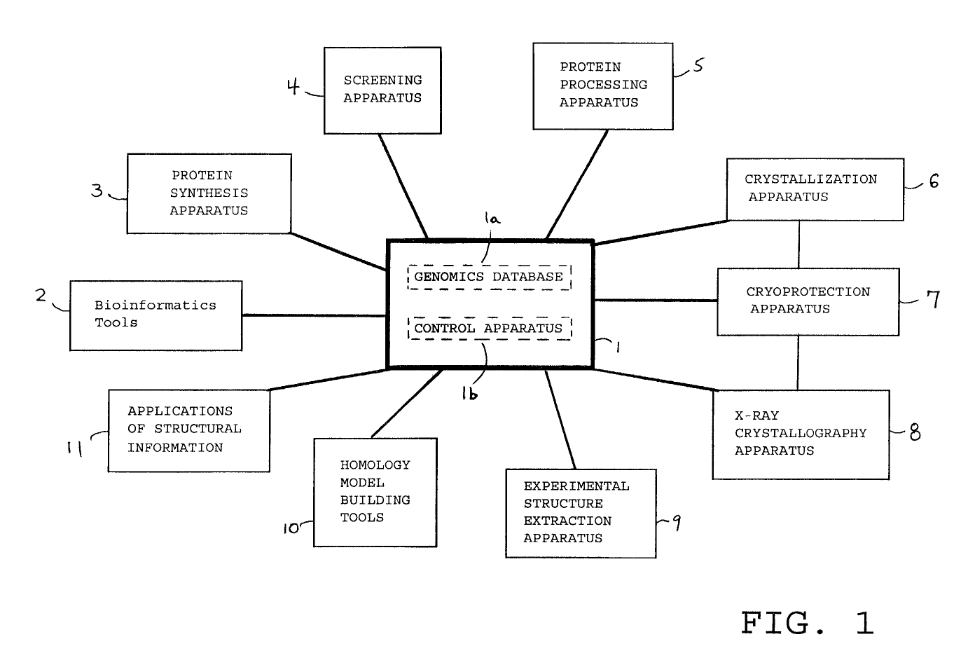

An embodiment of the present invention is described below, with reference to FIG. 1.

The initial part of the present invention is the development of a structural genomics database. Database 1a is built using known structural information, sequence information and functional information. Database 1a is systematically organized in a user friendly manner, and includes a user interface to make it easy to use, even for a novice of computer use.

While 3D structures form the centerpiece of the present invention, the database itself would contain far more information, including vast amounts of data which would be organized and analyzed in a way only made possible by the structural information available through the database. The database constitutes a complete genomics database system consisting of linked database and advanced analysis tools. As an example of one database structure, each gene may be associated with one or more families, with pointers to related genes and biochemical pathways, including structural information provided where available. For each gene family the information may include lists of family members across species, multiple sequence and structural alignments, evolutionary trees, conservation patterns and active site residues, link to biochemical pathways, and pharmaceutical assay information (such as binding data) on relevant drugs where available. Annotations may include electrostatic properties, physico-chemical characterization of binding surfaces and other functionally important regions, domain definition, evolutionary patterns, functional epitopes, derived pharmacophores, and ultimately, screened “virtual” libraries of small-molecule compounds. The database may be constructed to remain dynamic, with continuous updates of information items and relationships between items.

System component 1 includes database 1a and controller 1b which controls updates of database 1a. Controller 1b also provides control information to other components in the system. Database 1a is updated when newly acquired structural, sequence and functional information, including proprietary structures determined by the process and system of the present invention as well as information obtained from other sources, is received.

Three dimensional structural information may be exploited in conjunction with recent advances in amino acid sequence analysis to construct the database. Advanced bioinformatics tools 2 are used to cluster all known gene products into families of homologous sequences. The clustered gene products are typically similar at approximately 30% identity, <0.001 probability of error. The structure of a representative member for each and every family is determined. The protein classes may include whole proteins, domains or sequence motifs that may or may not correspond to independent modules. The unsolved members, which probably constitute the majority, of each family may be visualized by homology modeling based on the known structures of family representatives, as described below.

Sequence analysis programs such as BLAST as well as other tools may be used. The other tools may implement strategies such as (1) iterative cycles of sequence search and family identification, (2) profile search based on family analysis and (3) domain identification. These other tools may be used to expedite identification of remote sequence homologs. Some bioinformatics tools implement fold recognition methods which use structural information to identify relationships between proteins with very different sequences.

Bioinformatics tools 2 may include one or more computing systems running software containing computational solution techniques for analyzing genomic, structural and other biological data and information obtained by experiments, modeling, database search and instrumentation.

Once gene products have been organized into families, crystals are produced using a series of steps that includes (1) molecular cloning of the selected target, (2) protein expression, (3) biochemical purification, and (4) crystallization.

Component 3 is used to simultaneously, in parallel for each such family, synthesize member proteins using information of appropriately representative species. For example, protein synthesis unit 3 may be used to clone a few cDNAs from the representative species into expression vectors for a few expressions systems. Three to six cDNAs may be selected for cloning, and one to four expression systems may be used. A variety of expression systems may be established to include E. coli, baculovirus infected insect cells, Drosophila, Pichia yeast, and Chinese Hamster Ovary cells. Both cytoplasmic and secretion systems may be used as appropriate, with and without affinity tags. Because of its speed and economy, expression in E. coli may be emphasized and this includes urea extraction and refolding from inclusion bodies. E. coli expression is also advantageous for the ease of selenomethionine incorporation, which may be used routinely at the outset of production expression. Automation may be introduced wherever possible, including the cloning and expression steps.

Protein synthesis unit 3 alternatively may perform chemical synthesis of polypeptide followed by refolding into native proteins. Another possible alternative would be synthesis by means of in vitro translation or any other method by which protein may be synthesized.

Next, system component 4 is used to screen for expression the constructs resulting from the cloning. Component 4 determines the constructs that are effective, which then advance to the preparative step. Where possible, crystals may be screened on home equipment.

The expressed proteins identified by component 4 are prepared, purified and characterized using apparatus 5. Frequently, the preparative expression is prepared from the outset as the selenomethionyl analog to be used in structure determination by the multi-wavelength anomalous diffraction (MAD) phasing method, as described below. Each protein may be purified with affinity tags, and characterized for size, sequence authenticity, solubility, homogeneity and monodispersity. The purification function may be achieved in one step or in multiple steps. State-of-the-art chromatography and electrophoresis purifications, for example, may be used. The characterization function may be performed using any of a number of known techniques, including ultra-centrifugation, nuclear magnetic resonance spectroscopy, mass spectroscopy, and dynamic light scattering.

Apparatus 5 may comprise one or more physical units, each unit performing one or more of the preparation, purification and characterization functions. Data from the preparation, purification and characterization steps are supplied to controller 1b which supplies control information to apparatus 5.

Purified proteins processed with apparatus 5 are provided to crystallization apparatus 6. The purified proteins are set to crystallize in parallel against crystallization screens in crystallization apparatus 6. Next, the crystals that grow are tested for predetermined diffraction characteristics to determine the crystals that are suitable for diffraction measurements. Crystallization may use factorial designs in vapor diffusion set-ups generated by robotics.

Crystals determined by crystallization apparatus 6 to be suitable are supplied to and frozen in cryoprotection apparatus 7. Apparatus 7 typically uses flash freezing. Other cryoprotection techniques, however, may be used.

A frozen crystal is removed from cryoprotection apparatus 7 and supplied to X-ray crystallography apparatus 8. Apparatus 8 includes a synchrotron storage ring using undulator beamlines designed specifically for high-throughput crystallography. Appropriate electronic detectors of adequate size are used. The detectors may be 2k by 2k charge-coupled device (CCD) arrays. Pixel array, such as CMOS, or other advanced area detectors may be used in the alternative.

The analysis of crystal structure involves a series of steps, including (1) crystal characterization, (2) diffraction measurements, (3) phase determination, (4) density-map interpretation, and (5) structure refinement. The strategy of analysis may be closely integrated with the expression and synchrotron portions, including as a standard the incorporation of selenomethionine and MAD phasing on small frozen crystals. Most data may be measured at the synchrotron facility, but when feasible (such as for molecular replacement structures), home equipment may be used. Standard as well as specially developed computer programs may be used with a system of PC and workstation computers, preferably to graphically represent the information.

Diffraction data for the crystal are measured using apparatus 8 with the MAD method. Typically, this exploits the properties of Se from selenomethionyl proteins, but any one of several other heavy atoms can be used. Alternatively or in conjunction with the MAD experiments, analysis may include the method of multiple isomorphous replacement (MIR). Next, using apparatus 8, the diffraction data are analyzed with the MAD phasing method or by another technique, an atomic model is built, and the model is refined against the diffraction data. The refined model is stored in database 1a.

Apparatus 8 should be a facility optimized for high throughput macromolecular crystallography. The facility may include two undulator beamlines and one bending magnet beamline, such as can be implemented at one sector of the APS, subject to appropriate design within the abilities of one of skill in the art. Beamlines typically operate on the condition that a fraction of the beamline is supplied to independent investigators in order to recover some of the construction cost for the synchrotron. Typical experiments may take three days at a second generation source, but only a few hours at a third generation source such as the APS. Even at a ten-fold enhancement of throughput over facilities such as NSLS at Brookhaven, apparatus 8 may be used to produce as much as 400 novel proprietary structures per year, which is comparable to the current rate of production from the entire world, and more than double the production of truly novel results.

Four aspects of the capabilities of the APS make it a useful model as a facility. First, since high throughput is a priority, the markedly enhanced flux from APS undulators relative to conventional bending magnets is itself an important, even for currently typical protein crystals. In addition, the brightness of undulator radiation is essential for solving structures from samples that would otherwise be intractable. The brightness provides energy resolution, spatial resolution and angular resolution. The signals for MAD phasing depend on electronic transitions that often have sufficiently short lifetimes requiring high energy resolution (less than 2 eV) for optimization. This is rarely achieved in current practice, but the low intrinsic divergence from an APS undulator is a good match for narrow bandwidth monochromators. The ability to focus the entire undulator output into a very fine spot, such as under 50 by 100 microns, should make diffraction from microcrystals (smaller than 20 microns) very feasible. It is often much easier to obtain small crystals than to achieve growth to a larger size, and smaller crystals tend to be more perfect and to freeze more readily. Some of the molecules are likely to crystallize into large unit cells, such as having greater than 500 Å cell edges. Here again the low intrinsic divergence is of great use, and more generally provides for improved spatial resolution at the detector surface which would enhance data accuracy for nearly all problems.

The insertion device (ID) and bending magnet (BM) beamlines of one APS sector may be used. The BM beamline may include a single station for crystal characterizations and for data collections on strongly diffracting crystals. The ID beamline may include two experimental stations fed by tandem and independently tunable undulators. The end station may have optics similar to those for Structural Biology Center Collaborative Access Team beamlines at sector 19 of the APS and the side station may use diamond-crystal technology like that implemented at the TROIKA and QUADRIGA beamlines at the European Synchrotron Radiation Facility.

MAD experiments may be performed over a broad range of absorptive transitions in an accessible range of X-rays from approximately 3.5 to 35 keV. This includes K-edges from calcium to xenon (Z=20-54), L-edges from cadmium to uranium (Z=48-92), and the exceptionally powerful M-edges of uranium. The ID end station and BM beamline should permit this full range of experiments. Experiments at extremes of the full range are more difficult, however, and nearly all successful MAD applications to date have been in the range from the iron K-edge (7.1 keV) to the uranium L_{III}-edge (16.7 KeV). The beamline optics, within the constraints of other specifications, may be optimized for such experiments.

The geometry of the diamond-crystal side station necessarily constrains the accessible energy span. A constrained range from 10 to 14 keV nevertheless accommodates the heart of applications, including the important Se and Br K-edges and L_{III}-edges for the heavy metals from atomic number 74 to 83 (W, Re, Os, Ir, Pt, Au, Hg, Tl, Pb, Bi). In order to optimize the radiation for this span, a shorter-period undulator that would produce higher first-harmonic intensity throughout this range than that of the 3.3 cm period device should be used. Since MAD experiments require undulator gap adjustments and the diamond monochromator removes selected radiation from the downstream spectrum, a scheduling constraint may be imposed against simultaneous experiments at the same absorptive edge. Of course the bending magnet line can always operate independently.

The beamline optics and experimental apparatus must also be optimal for rapid and accurate diffraction experiments in support of MAD phasing on small crystals. Thus, beams are typically focussed to under 100 microns spheres of confusion. Beam divergences from undulators are intrinsically small. Monochromator crystals should be selected to provide high energy resolution. Detectors must have rapid read-out. CCD, pixel array, such as CMOS, or other advanced area detectors may be used.

Sample cooling is a concern and may require some experimentation. Whenever beams are overpowering for sample integrity, the philosophy will be to reduce power in ways that exploit brightness. Thus, apertures to select the heart of the beam and monochromators to give a fine bandpass should be used instead of attenuator filters.

Next, component 9 retrieves the refined model along with other information from database 1a, and analyzes the retrieved model while using sequence information of other family members and information of other known 3D structures. Analyzer 9 also analyzes the refined model for surface characteristics, such as electrostatic potential, hydrophobicity, curvature and variability, using a program such as GRASP, with the aim to define active sites and macromolecular contact sites. For relevant structures, component 9 defines classes of compounds predicted to have binding potency while using the information of active site properties. The class definitions are supplied to and stored in database 1a.

Computational tools 10 for homology model building are used to develop models for homologs. The atomic model of one family member is retrieved from database 1a, and used to predict a model of other useful family members. Provided that sequence similarities are sufficiently high (e.g., 50% identity), excellent models can be constructed by homology modeling methods. General characteristics of, for example, polypeptide folding can be modeled even when similarities are modes (ca. 30% identity).

Such atomic models are useful in, for example, medicine, agriculture and biotechnology. The homology models may be used in target selection or drug design. The models may also be used to design more appropriate constructs for experimental analysis of the human homolog. Thus, for example, an enzyme involved in cholesterol synthesis in humans could be a target for structure-based design of cardiovascular therapeutics provided that an appropriate atomic model is available. Even the structure of a related molecule from a bacterium might be useful as a guide for initial efforts. The models, constructed with the benefit of the structural database, may be used as the foundation for modeling techniques such as secondary structure prediction. Homology model building tools, like other components, typically comprises software which is run on a personal computer or workstation that may or may not be used for other functions in the system.

The ultimate goal is to obtain 3D atomic models for protein and RNA molecules representing all major expressed gene families. Structures for sub-family representatives, specific therapeutic target, and important homology models may also be included. As an initial step, bioinformatics may be used to choose crystallization targets and may assist in the construction of a pilot database derived from known 3D structures. However, the database would undergo constant change and revision as new data and new methods become available. The bioinformatics component selects targets for expression and crystallization, and assemble the results into the database. A synchrotron facility is used while parallel efforts in the expression of proteins for crystallization and in the analysis of diffraction results keep pace with the synchrotron.

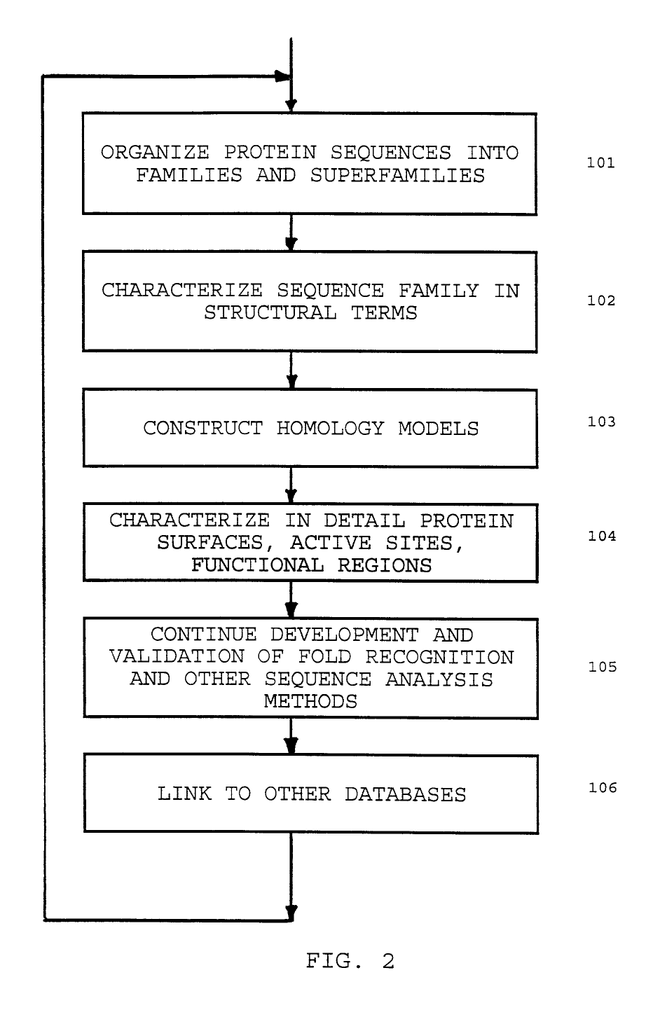

In a preferred process of the present invention which includes the following steps, as shown in FIG. 2, the steps are continuously iterated to develop a comprehensive structural genomics database. In step 101, protein sequences are organized into families and superfamilies, which is required initially for prioritizing crystallization targets. Next, in step 102, each sequence family is characterized in structural terms. In step 103, homology models are constructed. In step 104, protein surfaces, active sites, functional regions, etc., are characterized in detail. In step 105, development and validation of fold recognition and other sequence analysis methods is continued. In step 106, links to other databases which include biological pathways, functional annotation, and small molecules are generated.

At all steps of the process, parallel technology including robotics and other automation may be used. Subject materials may be monitored and logged at each step, and process control data of this kind may be used to optimize the procedures. Records maintained on subjects that do not advance may be used to reinitiate such experiments as advanced procedures are implemented.

The database has enormous commercial value to, for example, biotechnology, agriculture and the pharmaceutical industry. The structural information may be used in a number of ways. Some of the structures or related family members are likely to be drug targets and may be used directly for this purpose. The structures may also be used to provide a structure characterization of as many gene families as possible, while in parallel providing detailed structural coverage within gene families, focusing at an early stage, for example, on proteins of great pharmaceutical interest such as kinases or helical cytokines. More extensive coverage within families would allow the construction of more accurate homology models. Another protein family of major importance is the family of G protein coupled receptors. While none of these membrane proteins has yet been crystallized, given the intense efforts being devoted to this problem in labs around the world, it is likely that a breakthrough will be reported within the next few years. If and when this occurs, the present invention would provide a tool for quickly solving the structures of a large number of these proteins which are important pharmaceutical targets.

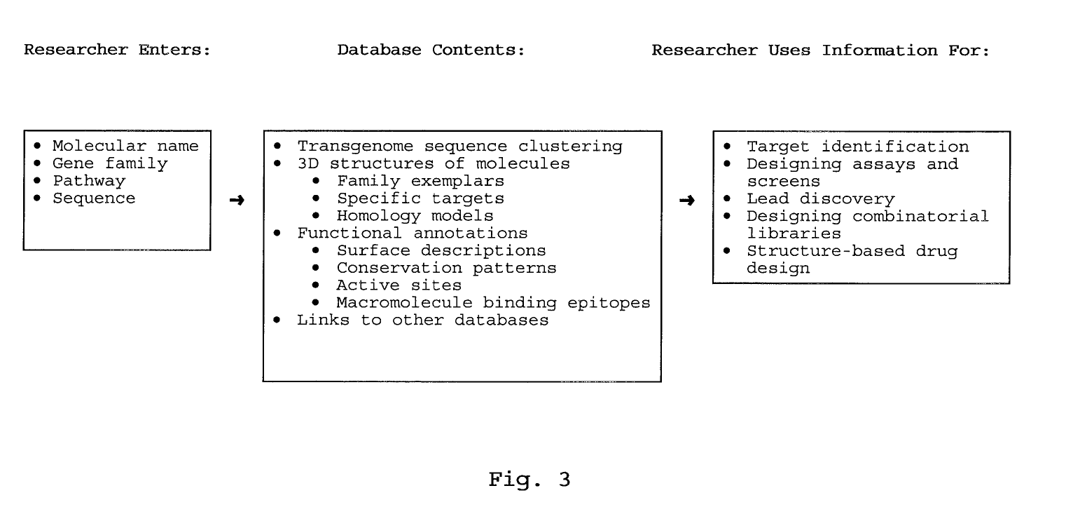

The assembled information system enables efficient search of the database for new drug targets and their functional annotation. In one embodiment, as shown in FIG. 3, users may access and browse through the database by entering descriptors such as a molecular name, a gene family name, a protein family or protein name, a metabolic pathway name or a particular sequence. The preferred access route would be through partial and full-length sequences. The typical scientist in a pharmaceutical company would have immediate and convenient access to all available information on a list of sequences of interest obtained from, for example, external sources. The database reduces the need for in-house expertise in sequence analysis because the results of the most advanced type of such analysis is contained in the database. More importantly, the fact that the database contains, and exploits, a large number of 3D structures, some possibly not publicly available, would provide the user a significant competitive advantage in the process of target identification.

A second application is in structure-based drug design. Three dimensional structural information may be used to specify the characteristics of peptides and small molecules that might bind to or mimic a target of interest. These descriptors may then be used to search small molecule databases and to establish constraints for use in the design of combinatorial libraries. As with target identification, the structural information may be used in a feedback loop involving experimental tests.

The linkage of the database with screening data and small molecule data available in pharmaceutical and biotechnology companies would enable a continuous interaction amongst experiments that identify gene sequences (i.e. from chip technologies), protein structures and chemical libraries. The impact on the drug discovery process may be enormous.

While an embodiment of the invention has been described in detail, it should be understood that the invention is not limited to that precise embodiment and that various changes and modifications thereof could be effected by one skilled in the art without departing from the spirit or scope of the concepts of the invention recited in the appended claims. For example, for simplicity, the above has been described with only proteins, but it would be apparent to one skilled in the art that the principles also apply to RNA, and changes and modifications to the embodiment could be effected by one skilled in the art to practice the invention with RNA without undue experimentation.

Disclosures of the following publications in their entireties are hereby incorporated by reference into this application to more fully describe the state of the art to which this invention pertains:

W. A. Hendrickson, J. R. Horton and D. M. LeMaster, “Selenomethionyl Proteins Produced for Analysis by Multiwavelength Anomalous Diffraction (MAD): A Vehicle for Direct Determination of Three-Dimensional Structure,” EMBO J., 9:1665-1672(1990).

W. Yang, W. A. Hendrickson, R. J. Crouch and Y. Satow, “Structure of Ribonuclease H Phased at 2 Å Resolution by MAD Analysis of the Selenomethionyl Protein,” Science, 249:1398-1405 (1990).

W. A. Hendrickson, “Determination of Macromolecular Structures from Anomalous Diffraction of Synchrotron Radiation,” Science, 254:51-58(1991).

K. C. Smith, B. Honig, “Evaluation of the Conformational Free Energies of Loops in Proteins,” Proteins, 18:119-32 (1994).

B. Honig, A. Nicholls, “Classical Electrostatics in Biology and Chemistry,” Science, 268:1144-49 (1995)

L. Shapiro, A. M. Fannon, P. D. Kwong, A. Thompson, M. S. Lehmann, G. Grubel, J. F. Legrand, J. Als-Nielsen, D. R. Colman, W. A. Hendrickson, “Structural Basis of Cell-Cell Adhesion by Cadherins,” Nature, 374:327-37 (1995).

N. Ben-Tal, A. Ben-Shaul, A. Nicholls, B. Honig, “Free-energy Determinants of Alpha-helix Insertion into Lipid Bilayers,” Biophys J, 70:1803-12 (1996).

N. Froloff, A. Windemuth, B. Honig, “On the Calculation of Binding Free Energies Using Continuum Methods: Application to Mhc Class I Protein-peptide Interactions,” Protein Sci, 6:1293-301 (1997).

W. A. Hendrickson and C. M. Hendrickson, “Phase Determination by the Method of Multiwavelength Anomalous Diffraction (MAD),” Methods in Enzymology, 276:494-523 (1997).

B. Honig, “New Challenges in Computational Biochemistry,” Pac Symp Biocomput, 21-24 (1997).

C. D. Lima, K.L. D'Amico, I. Naday, G. Rosenbaum, E. M. Westbrook, W. A. Hendrickson, “MAD Analysis of FHIT, a Putative Human Tumor Suppressor from the HIT Protein Family,” Structure, 5:763-74 (1997).

L. Shapiro and C. D. Lima, “The Argonne Structural Genomics Workshop: Lamaze Class for the Birth of a New Science,” Structure, 6:265-67 (1998).

W. A. Hendrickson, H. Wu, J. L. Smith, W. I. Weis, et al., “MADSYS, a Computer System for Phase Evaluation from Measurements of Multiwavelength Anomalous Diffraction”.

The following computer programs are hereby incorporated by reference into this application to more fully describe the state of the art to which this invention pertains:

Information regarding the GRASP program mentioned hereinabove may be obtained at the following Web address: “http://honiglab.cpmc.columbia.edu/grasp/”. The GRASP program can be licensed from Columbia University. Information regarding licensing of GRASP from Columbia University may be obtained at the following Web address:

“http://honiglab.cpmc.columbia.edu/grasp/G_academic.html”.

Information regarding the MADSYS software and information regarding how to obtain a copy of MADSYS may be obtained at the following Web address:

“http://convex.hhmi.columbia.edu/hendw/madsys/madsys.html”.

What is claimed is:

1. A system for pan-genomic determination of three-dimensional macromolecular atomic structures, comprising:
database means for systematically organizing all known structural information into a genomics database of structural information, sequence information and functional information;
bioinformatics means for using said structural information, sequence information and functional information stored in said database to cluster a plurality of known gene products into a plurality of families of homologous sequences;
protein synthesis means for synthesizing for each family, in parallel simultaneously, a plurality of member proteins using genomics information of a plurality of appropriately representative species;
screening means for screening the synthesized proteins to determine ones that are effective;
protein processing means for preparing, purifying and characterizing the screened proteins determined to be effective by said screening means;
crystallization means for crystallizing a plurality of the purified proteins in parallel against a plurality of crystallization screens, and testing a plurality of grown crystals for predetermined diffraction characteristics to determine suitable ones of said plurality of grown crystals;
X-ray crystallography means for performing high-throughput crystallography, said X-ray crystallography means having diffraction measuring means for measuring a suitable crystal for diffraction data, analyzing means for analyzing said diffraction data, means for building an atomic model, and means for refining said model against said diffraction data and storing the refined model in said database;
structure extraction means having means for analyzing the refined model using sequence information of other family members and information of other known three-dimensional structures, means for analyzing for functional motifs and for surface characteristics to define active sites and macromolecular contact sites, and means for defining at least one class of compounds predicted to have binding potency using the active sites information; and
homology model building means for developing a homology model using an atomic model retrieved from said database,
wherein an ensemble of all known structures is used to further advance an effectiveness of said bioinformatics means.

2. A system according to claim 1, further comprising:
cryoprotection means for freezing said suitable ones of said plurality of crystals which are supplied by said crystallization means,
wherein said suitable crystal is frozen by said cryoprotection means before being measured for said diffraction data by said diffraction measuring means.

3. A system according to claim 1, wherein
said protein synthesis means includes cloning means for cloning for said each family, in parallel simultaneously, cDNAs of said plurality of appropriately representative species into a plurality of expression vectors for a plurality of expressions systems,
said screening means screens for expression constructs obtained from said cloning means to determine ones that are effective, and
said protein processing means processes the expressed proteins determined to be effective by said screening means.

4. A system according to claim 1, wherein
said X-ray crystallography means includes a synchrotron storage ring having undulator beamlines for high-throughput crystallography by a multiwavelength anomalous diffraction method, and
said analyzing means analyzes said diffraction data by a multiwavelength anomalous diffraction phasing method.

5. A system according to claim 4, wherein selenomethionine is incorporated in the synthesized proteins by said protein synthesis means, and said analyzing means using said multiwavelength anomalous diffraction phasing method analyzes said diffraction data of selenomethionyl proteins.

6. A system according to claim 1, wherein said homology model is used in at least one of target selection, drug design, and design of more appropriate constructs for experimental analysis.

7. A process for pan-genomic determination of threedimensional macromolecular atomic structures, comprising the steps of:
systematically organizing all known structural information, sequence information and functional information into a genomics database;
clustering a plurality of known gene products into a plurality of families of homologous sequences using at least one bioinformatics tools;
synthesizing for each family, in parallel simultaneously, a plurality of member proteins using genomics information of a plurality of appropriately representative species;
screening the synthesized proteins to determine ones that are effective;
preparing, purifying and characterizing the screened proteins determined to be effective in said step of screening;
crystallizing a plurality of purified proteins in parallel against a plurality of crystallization screens;
testing a plurality of grown crystals for predetermined diffraction characteristics to determine suitable ones of said plurality of grown crystals;
performing high-throughput crystallography, including measuring a suitable crystal for diffraction data, build an atomic model according to an analysis of said diffraction data, refining said model against said diffraction data, and storing the refined model in said database;
analyzing the refined model using sequence information from other family members and information of other known three-dimensional structures, analyzing for functional motifs and for surface characteristics to define active sites and macromolecular contact sites, and defining at least one class of compounds predicted to have binding potency using the active sites information; and
developing a homology model using computational tools for homology model building and an atomic model retrieved from said database,
wherein said database is updated by using said bioinformatics tools along with said all known structural information and an ensemble of all known structures is used to further advance an effectiveness of said bioinformatics tools.

8. A process according to claim 7, further comprising the step of:
freezing selected ones of said plurality of grown crystals determined to be suitable in said step of testing,
wherein said suitable crystal is frozen before being measured for said diffraction data in said step of measuring.

9. A process according to claim 7, wherein
said step of synthesis includes the step of cloning for said each family, in parallel simultaneously, cDNAs from said plurality of appropriately representative species into a plurality of expression vectors for a plurality of expressions systems,
constructs obtained in said step of cloning are screened for expression to determine ones that are effective, and
the expressed proteins determined to be effective are processed in said step of preparing, purifying and characterizing.

10. A process according to claim 7, wherein
said high-throughput crystallography is performed using a synchrotron storage ring having undulator beamlines along with a multiwavelength anomalous diffraction method, and
said diffraction data is analyzed using a multiwavelength anomalous diffraction phasing method.

11. A process according to claim 10, wherein selenomethionine is incorporated in the synthesized proteins, and said multiwavelength anomalous diffraction phasing method is used to analyze said diffraction data of selenomethionyl proteins.

12. A process according to claim 7, further comprising the step of
using said homology model in at least one of target selection, drug design, and design of more appropriate constructs for experimental analysis.
